(19) United States
(12) Patent Application Publication
(10) Pub. No.: US 2025/0049495 A1
(43) Pub. Date: Feb. 13, 2025

(54) ELECTROSURGICAL DEVICE

(71) Applicant: Cilag GmbH International, Zug (CH)

(72) Inventors: Barry C. Worrell, Centreville, OH (US); Roopesh Kumar, Cincinnati, OH (US); Ruben Salazar, Cincinnati, OH (US)

(21) Appl. No.: 18/446,222

(22) Filed: Aug. 8, 2023

(51) Int. Cl. A61B 18/14

(52) U.S. Cl. CPC A61B 18/1445; A61B 2018/00428; A61B 2018/0063; A61B 2018/1455

(57) ABSTRACT

Electrosurgical devices and methods associated therewith are provided. The electrosurgical devices include a proximal end, a shaft extending distally from the proximal end, and an end effector operatively coupled to a distal end of the shaft. The shaft consists of a monolithic outer tube, and a single inner guide disposed within the monolithic outer tube and extending a length thereof. The end effector includes a pair of jaws configured to grasp tissue and to receive a blade from the shaft to transect the grasped tissue.

### FIELD

Electrosurgical devices having an improved shaft design.

### BACKGROUND

Various electrosurgical devices, including vessel sealers, etc., have been developed that include components used to grasp, transect, and/or staple tissue during surgical operations, as well as to generate and transmit RF energy (e.g., monopolar and/or bipolar energy) to tissue in order to coagulate or seal the tissue as desired. One example of such an electrosurgical device is the ENSEAL® Tissue Sealing Device by Ethicon Endo-Surgery, Inc., of Cincinnati, Ohio.

These electrosurgical instruments typically include a handpiece, a shaft extending distally from the handpiece, and an end effector (often in the form of a pair of jaws) disposed at a distal end of the shaft. The handpiece can include inputs, such as a pivoting grip, trigger, and/or buttons and other inputs, which can be used to carry out operations using the end effector during a surgical procedure and as desired by an operator. However, due to the variety of operations capable of being performed by such instruments, many components must operate within the confines of the shaft in a coordinated fashion in order to translate inputs at the handpiece to operations at the end effector. The constraints of the shaft, as well as the demands placed upon it, can cause unwanted motion, articulation, and stress, each of which reduce the precision afforded to operators of the electrosurgical instruments during procedures, especially among procedures that require fine motor coordination.

Accordingly, there remains a need to improve the shaft design in such instruments in order to mitigate surgical errors and harm arising from such errors.

### SUMMARY

Electrosurgical devices, including vessel sealers, etc., with improved shaft designs, and related methods are provided.

In an embodiment, an electrosurgical device is provided. The electrosurgical device can include a proximal end, a shaft extending distally from the proximal end, an outer guide coupled to the shaft and the proximal end, and an end effector operatively coupled to a distal end of the shaft. The shaft can consist of a monolithic outer tube and a single inner guide disposed within the monolithic outer tube and extending a length thereof. The outer guide can be configured to minimize unwanted motion between the proximal end and the shaft. The end effector can include a pair of jaws configured to grasp tissue and to receive a blade from the shaft to transect the grasped tissue.

The electrosurgical device can vary in a number of ways. For example, the singular inner guide can include a plurality of channels defined in an exterior thereof. Each of the channels within the plurality of channels can extend an entire length of the singular inner guide and can be configured to receive an element in electrical communication with the proximal end. In some variations, the plurality of channels can include an active rod channel, ground wire channel, and/or a knife track. The active rod channel can be configured to receive an active rod therein, and can include a proximal ramp feature configured to direct the active rod received within the active rod channel toward a center of the singular inner guide. The knife track can be configured to prevent buckling of a knife positioned therein during an activation stroke of the knife. In another example, the monolithic outer sleeve can have a tapered hole therein and the singular inner guide can have a second tapered hole therein. The first and second tapered holes can be configured to align when the singular inner guide is properly positioned within the monolithic outer sleeve. In another example, the electrosurgical device can include heat shrink tubing (or insulative coating) disposed around the shaft. In another example, the monolithic outer guide can include a central lumen configured to receive the shaft therethrough. The monolithic outer guide can include an outer guide clip configured to couple to the monolithic outer guide and to the shaft, and configured to prevent rotation of the shaft relative to the monolithic outer guide. In some variations, the outer guide clip can be substantially Y-shaped. In other variations, the outer guide clip can be configured to couple to corresponding grooves in an exterior of the shaft.

In another embodiment, an electrosurgical device is provided. The electrosurgical device can include a proximal end, a shaft extending distally from the proximal end, and an end effector operatively coupled to a distal end of the shaft. The shaft can be secured to the proximal end via an outer guide. The outer guide can include an outer guide base, an outer cover, and an outer guide clip configured to couple the outer guide to the shaft. The shaft can consist of a monolithic outer tube, and a single inner guide disposed within the outer tube. The end effector can include a pair of jaws configured to grasp tissue. The outer guide can be configured to minimize user independent motion between the proximal end and the shaft.

The electrosurgical device can vary in a number of ways. For example, the singular inner guide can include a plurality of channels defined in an exterior thereof, and each of the channels within the plurality of channels can extend an entire length of the singular inner guide and can be configured to receive an element in electrical communication with the proximal end. In some variations, the plurality of channels can include an active rod channel and/or ground wire channel defined in an exterior thereof, and the singular inner guide can a proximal ramp feature configured to direct an active rod received within the active rod channel toward a center of the singular inner guide. In other variations, the electrosurgical device can include a knife disposed in the plurality of channels, which can be configured to extend into the pair of jaws to transect grasped tissue during an activation stroke triggered by an input at the proximal end. In further variations, the electrosurgical device can include an active rod extending between the handpiece and the end effector, which can be configured to conduct an electrical current generated by the handpiece to tissue grasped in the pair of jaws. In still further variations, the plurality of channels can include a knife track being configured prevent buckling of a knife positioned therein during an activation stroke of the knife. In another example, the monolithic outer sleeve can have a tapered hole therein and the singular inner guide can have a second tapered hole therein. The first and second tapered holes can be configured to align when the singular inner guide is properly positioned within the monolithic outer sleeve. In another example, the electrosurgical device can include heat shrink tubing disposed around the shaft.

The electrosurgical device can vary in a number of ways. For example,

The electrosurgical device can vary in a number of ways. For example,

### BRIEF DESCRIPTION OF THE DRAWINGS

The invention will be more fully understood from the following detailed description taken in conjunction with the accompanying drawings, in which:

FIG. 1 is a simplified side view of an exemplary electrosurgical device;

FIG. 2 is an exploded view of an electrosurgical device, including an outer guide assembly, according to an embodiment;

FIG. 3 is a partial exploded view of the outer guide assembly of FIG. 2;

FIG. 4 is a cross-sectional view of the electrosurgical device of FIG. 1;

FIG. 5 is an exploded view of the shaft and outer guide assembly of the electrosurgical device of FIG. 1 in a deconstructed state;

FIG. 6 is a perspective view of the shaft and the outer guide assembly of FIG. 5 in a constructed state;

FIG. 7 is a partial perspective view of the electrosurgical instrument of FIG. 1, including a distal end of an active rod channel defined within a singular inner guide of the shaft;

FIG. 8 is a partial perspective view of a proximal end of the active rod channel of FIG. 7;

FIG. 9 is a partial perspective view of the shaft of FIG. 6 with an adapter portion;

FIG. 10 is a partial perspective view of the shaft and adapter portion of FIG. 9;

FIG. 11 is a partial top view of the shaft of FIG. 1 during rotational alignment;

FIG. 12 is a partial side view of the shaft of FIG. 1 during axial alignment;

FIG. 13 is a partial perspective view of the shaft of FIG. 6 with a knife channel;

FIG. 14 is a partial perspective view of the singular inner guide of FIG. 7, including the knife channel of FIG. 13; and

FIG. 15 is a partial perspective view of the singular inner guide of FIG. 7, including a closure band channel.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

The present disclosure is generally directed to improved designs for electrosurgical instruments, including vessel sealers. These instruments typically include a proximal end portion, such as a handpiece, and a shaft extending distally from the proximal end portion. In prior designs, multiple components were used to make the shaft, as well as the interface between the proximal end portion and the shaft, which was relatively expensive and time consuming. If more than one manufacturer of the components were required, costs could be added as a result of the more complicated supply chain required to completely manufacture and distribute one instrument. Further, manufacturing variances in all of those components could contribute to an increased difficulty in performing precise surgical movements. This difficulty could be attributed to unwanted motion in the shaft relative to the proximal end portion, as well as unwanted bending and flexing of the shaft itself. The present design addresses the shortcomings of the prior art by reducing the number of components in the shaft, as well as in the interface between the proximal end portion and the shaft, while also adapting the interface design to minimize unwanted relative motion between the proximal end portion and the shaft. These improvements reduce manufacturing time and costs, simplify supply chain issues, and improve the overall operational accuracy. These improvements will be described in greater detail herein.

As introduced above, electrosurgical instruments and other instruments, such as, vessel sealers, etc., can be used for a variety of operations during a surgical procedure. These operations include: grasping tissue, transecting tissue, or otherwise mechanically joining tissue, coagulating tissue, sealing or otherwise energetically joining tissue, and more. Such instruments typically include a handpiece or a suitable base element for mating with a robotic surgical platform, a shaft extending from the handpiece (or base), and an end effector positioned at a distal end of the shaft, and inputs made at or on the handpiece (or base) can be translated into action by the end effector or proximate the end effector. While the term handpiece is used herein, instruments can be used with other systems, including robotic surgery systems or general telesurgical systems, where the instrument is manipulated by means other than the literal hands of an operator. A more general term, such as proximal end, can be used to refer to a portion of the instrument from which the shaft extends. Accordingly, the terms handpiece and base should not be construed to be limiting, and any of the concepts described herein are equally applicable to other types of systems which do not involve receiving inputs directly from a surgeon at a proximal portion of the instrument.

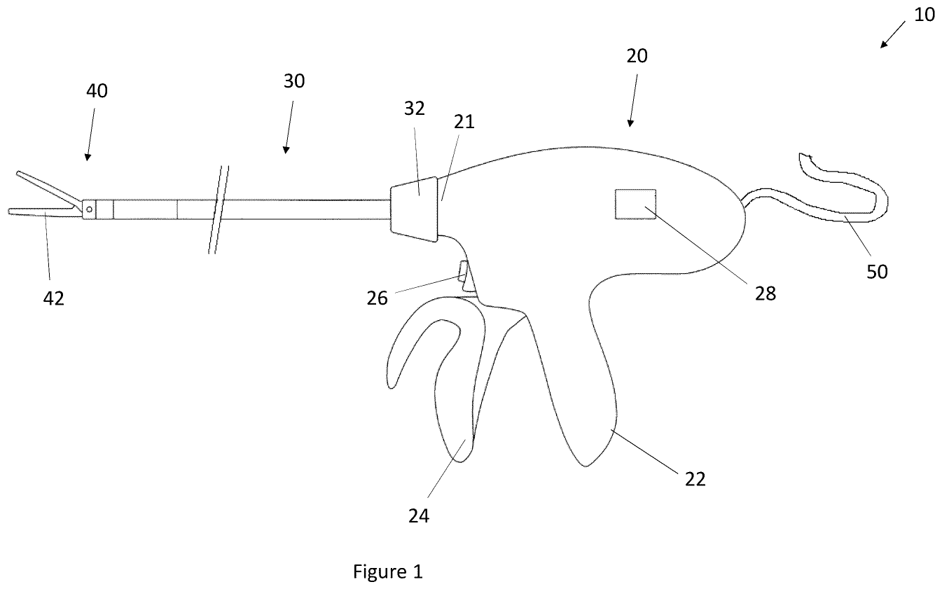

An exemplary instrument 10 can be seen in FIG. 1. The instrument 10 includes a handpiece 20, a shaft 30 extending distally from the handpiece 20, and an end effector 40 located at a distal end of the shaft 30. The handpiece 20, as depicted, includes a pistol grip 22 extending in a proximal direction from a bottom of the handpiece 20, a pivoting trigger 24 extending in a distal direction from a bottom of the handpiece 20, one or more activation inputs disposed at various locations thereon, including a trigger activation 26 as well as other inputs 28. The end effector 40 can include a pair of jaws 42 that can be used to perform many of the operations described herein. In all, the instrument 10 can be powered via a wired connection 50 and/or via a another kind of power system, such as a battery (not pictured). Certain components of the instrument 10 will be described in greater detail below.

In operation, a surgeon, operating the end effector, can manipulate the handpiece 20 and the inputs thereon at the proximal end of the instrument 10, mechanical and/or electrical inputs can be transferred through the shaft 30, and the desired operation (as listed above) can be carried out by the end effector 40. Moreover, in certain instrument designs, the shaft is articulable in pitch, yaw, and/or roll directions, and after articulation, operations can still be carried out in the articulated state. For example, the instrument 10 can feature a knob 32 located at the interface between the handpiece 20 and the shaft 30. The knob 32, in some variations, can be rotatable about a longitudinal axis of the shaft 30, which can cause the shaft 30 to rotate clockwise and/or counter-clockwise relative to the handpiece 20. This rotation, in turn, can cause the end effector 40 to rotate with the shaft 30 and the knob 32 and relative to the handpiece 20, which can provide certain advantages for an operator of the end effector during surgical procedures. These advantages can include providing a larger degree of maneuverability when manipulating tissue, for example. Improvements for instruments, including the instrument 10 featured in FIG. 1, are described herein, including improvements for the shaft 30.

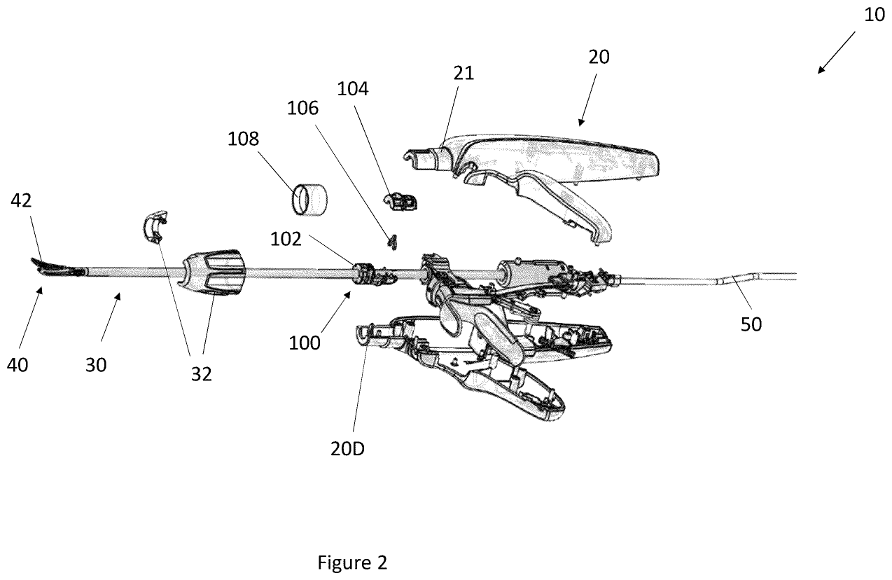

FIGS. 2-4 depict an outer guide 100 usable with the instrument 10. The interface between the distal portion of the handpiece 20—called the shroud 21—and the shaft 30 plays a key role in the looseness of the shaft within the handpiece 20, which can impact precision of operations in which the shaft is placed under lateral stress. Certain stress can cause the shaft 30 to flex in an unwanted manner, which can make precise operations more difficult, especially in prior art designs featuring an inferior interface. The outer guide 100 securely holds the shaft 30 within the shroud 21, which provides improvements over prior designs.

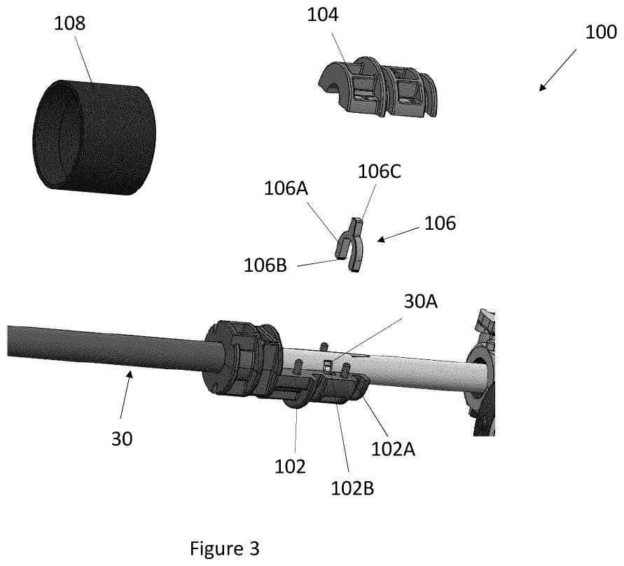

FIG. 2 depicts an exploded view of the instrument 10 featuring the outer guide 100. As depicted, the components generally include the end effector 40, the shaft 30, and the handpiece 20. Components of the outer guide 100 can be seen depicted as well, and these components are highlighted in greater detail in FIG. 3. These components can include an outer guide base 102, an outer guide clip 106, and an outer guide cap 104, each of which is depicted in FIGS. 2 and 3. These components can be made from a variety of materials including plastics, metals, composites, and/or other materials alone or in combination. Also depicted is a shroud ring 108, which can play a role in securing the shaft 30 to the shroud 21 in conjunction with the outer guide 100, as will be described in greater detail below.

The outer guide base 102 can be a monolithic element or comprising only a few elements, in contrast to prior art designs, which feature multiple elements in the same or a similar role. Additionally, the outer guide base 102 can have a generally cylindrical form defining a central lumen within which the shaft 30 can be received. A proximal portion of the outer guide base 102, seen especially in FIG. 3, can be in the form of a half cylinder. When the outer guide 100 is fully assembled, the outer guide cap 104—also in the form of a half cylinder and defining a portion of the central lumen to receive the shaft 30—can be coupled to the outer guide base 102 at this proximal portion to fully form the cylindrical shape of the two components. The outer guide cap 104 can be coupled to the outer guide base 102 in a number of ways, including, for example, via protrusions 102A located on the on the outer guide base 102, which can be sized to be received by complimentary recesses (not shown) located on the outer guide cap 104. Other means of joinder can be used as well, including a snap fitting, adhesive(s), welds, or other methods known in the art, as well as combinations thereof.

The outer guide clip 106, depicted in FIGS. 2 and 3 as a substantially y-shaped or “wishbone” shaped component, makes up part of the outer guide 100 as well. The outer guide clip 106 can include a pair of legs 106A defining opposed inner surfaces that are substantially flat and that together form a shaft seat 106B. The outer guide clip 106 can also include an upper stem 106C. While the outer guide clip 106 is not a perfect y-shape, it is substantially y-shaped in the sense that it includes the pair of legs 106A and the upper stem 106C—three protrusions extending generally from a central region of the outer guide clip 106—which, taken together, form a substantially y-shaped component. The upper surface of the proximal portion of the outer guide base 102 can include recesses 102B, which can be sized to receive portions of the outer guide clip 106, such as the legs 106A, in order to secure the outer guide clip 106 thereto. For example, as seen in the variation depicted in FIG. 3, the outer guide base 102 includes recesses 102B, which receive the legs 106A of the guide clip 106. A carve-out 30A of the shaft 30 proximate to the recesses 104B is present so that the legs 106A and the shaft seat 106B can be slid into position in order to secure the shaft 30 within the shaft seat 106B while the outer guide clip 106 is simultaneously secured to the outer guide base 102. In this way, an inner portion of the legs 106A are located within the shaft 30. When the outer guide clip 106 is secured to both the shaft 30 and the outer guide base 102, the outer guide 100 is prevented from rotating relative to the shaft 30. Moreover, the securement of the outer guide clip 106 can prevent undesired translational movement when the instrument 10 is clamped onto tissue, which assures that clamp force pressure is maintained when the jaws 42 are closed.

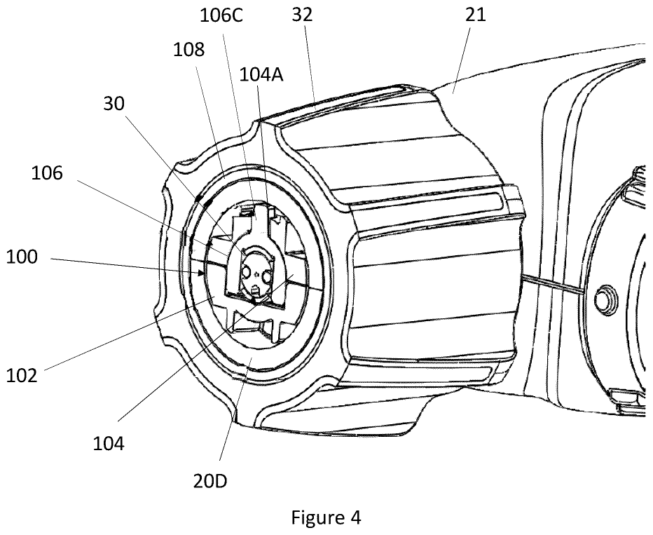

Further, the outer guide cap 104 can include a recess 104A located on an underside thereof, which can be sized to receive and engage the upper stem 106C of the outer guide clip 106. The recess 104A can be seen in cross-section in FIG. 4, for example, and seating the upper stem 106C. This can provide a more distributed loading profile and surface area, which can result in a lower stress on the outer guide 100 as whole. The upper stem 106C can extend through the top of the outer guide cap 104 for both assembly error proofing by providing a quick visual indication of proper placement, and to also provide a slight interference fit therebetween so that the outer guide clip 106 can be inserted into the outer guide cap 104 before assembling the outer guide base 102 and outer guide cap 104. When in proper position, the outer guide cap 104 will be engaged to both the outer guide base 102 and the outer guide clip 106. In this way, the outer guide assembly is able to minimize user-independent motion. In general, user-independent motion is motion not purposefully effected through intentional articulation of the instrument 10. For example, where a user or operator intentionally controls the instrument 10 to articulate in a certain way, that corresponding motion is user-dependent. However, where the instrument 10 experiences strain, bending, oscillation, or other such motion that occurs independent of whether the user or operator commanded such motion, that is the so-called user-independent motion. This user-independent motion introduces a level of uncertainty into finer movements and operations, which could contribute to operational errors and harm.

When the outer guide 100 is fully formed and secured to the shaft 30, the outer guide 100 can be located within the distal end 20D of the shroud 21, as seen in FIG. 2, for example. To further secure the outer guide 100 in position within the distal end 20D, an exterior of the outer guide 100 and an interior of the distal end 20D can include complementary elements, such as grooves and recesses, which complement each other in form and shape, and which can be coupled together to prevent certain movements of the outer guide 100 within the distal end 20D of the shroud 21. These features can be seen along the exterior of the outer guide base 102 and the outer guide cap 104 in FIGS. 2 and 3, as well as along the interior of the distal end in FIG. 2. Where deliberate rotational movement of the shaft 30 is desired, such as for additional articulation as described above, the grooves and recesses of the outer guide 100 and the distal end 20D of the shroud 21 can be designed to permit such rotation, as well as being designed to restrict such rotational motion to a certain range of motion. After the outer guide 100 is secured to the distal end 20D of the shroud 21, the shroud ring 108 can be positioned over the distal end 20D of the shroud 21 in order to clamp the components together. From there, the knob 32 can be positioned over the shroud ring 108 and/or part of the shroud 21 itself. A cross section of the knob 32, the shroud ring 108, the outer guide 100, and the shaft 30 can be seen in FIG. 4.

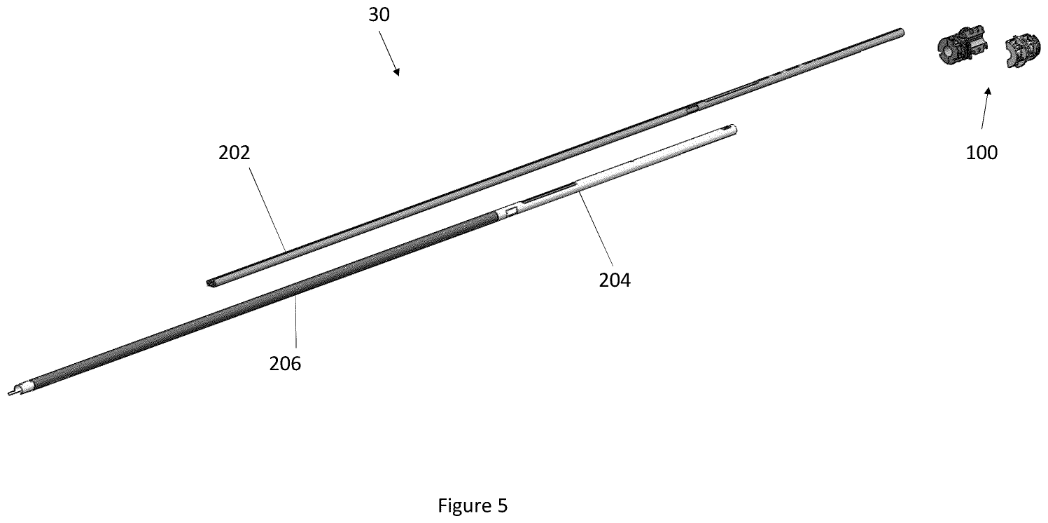

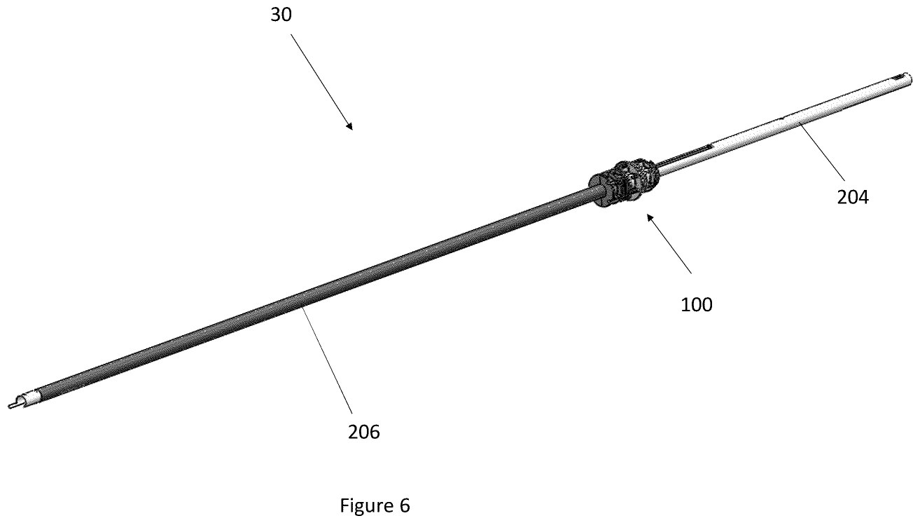

In addition to the inclusion of the outer guide 100, the shaft 30 itself can be improved over prior art designs to include features that contribute to an improvement in design, a reduction in the number of parts required, and increased performance. The shaft 30 can be seen in greater detail in FIGS. 5 and 6.

Generally, the shaft 30 can include a monolithic outer tube 204, and a singular inner guide 202. In some variations, heat shrink tubing 206 can be disposed around at least a portion of the monolithic outer tube 204, which, as the name suggests, can be a single unitary element. The singular inner guide 202 can be disposed within the monolithic outer tube 204, and, where included, the heat shrink tubing 206 can be placed around the monolithic outer tube 204 and singular inner guide 202 together. Also seen in FIGS. 5 and 6 is the outer guide 100, described above. The distal end of the outer guide 100 has an inner diameter region that fits tightly with the outer diameter of the monolithic outer tube 204. This fitting prevents high stresses in the more proximal region of the outer tube 204 by acting as a strain relief during shaft bending that may occur during surgical tasks, e.g., lifting tissue, manipulation of organs like the large intestine, etc.

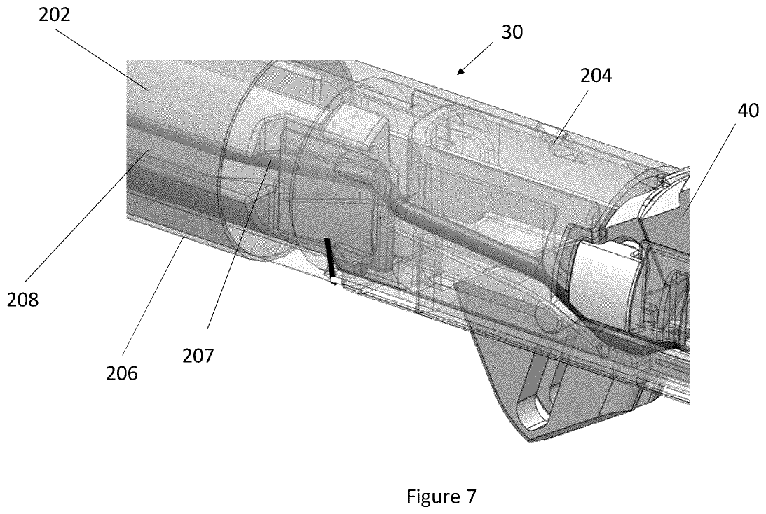

The singular inner guide 202 can be made of a variety of materials, such as plastics, metals, composites, ceramics, and more, as well as combinations thereof. The singular inner guide 202 can be made using a variety of processes, such as injection molding, machining, casting, or other known processes suitable to work the listed materials. Generally, the singular inner guide 202 can include a number of channels, which can be used to guide components through the shaft 30 as they are needed to perform operations with the instrument 10. These channels, as well as other features, will be described in reference to FIGS. 7-13. While the channels are depicted in specific locations on and/or within the singular inner guide 202, these specific locations are exemplary only and the actual locations and/or forms of the channels can vary depending upon the demands of the system.

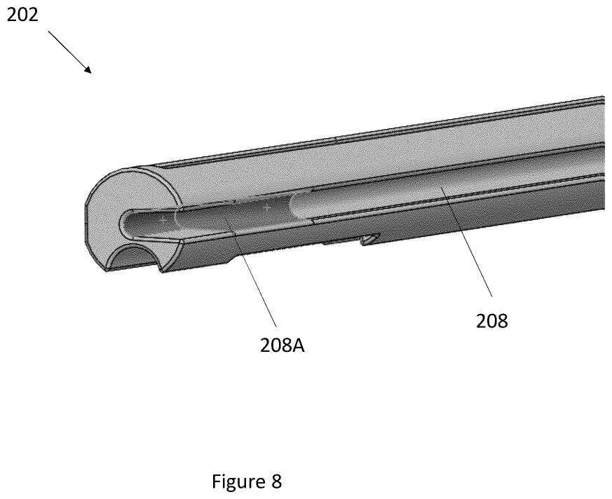

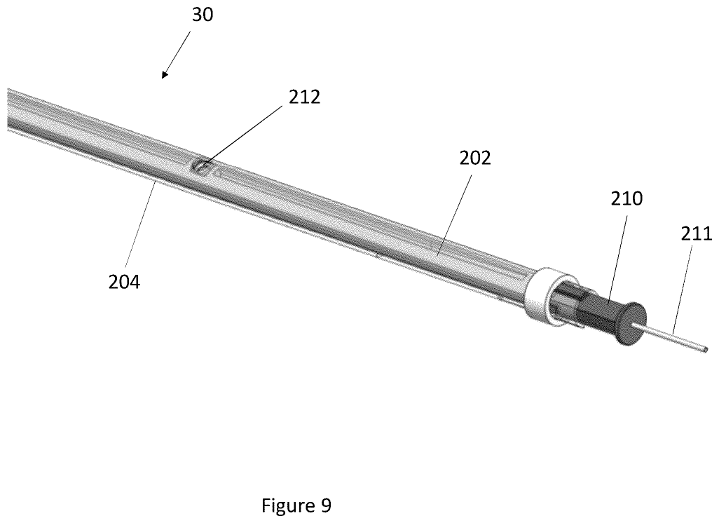

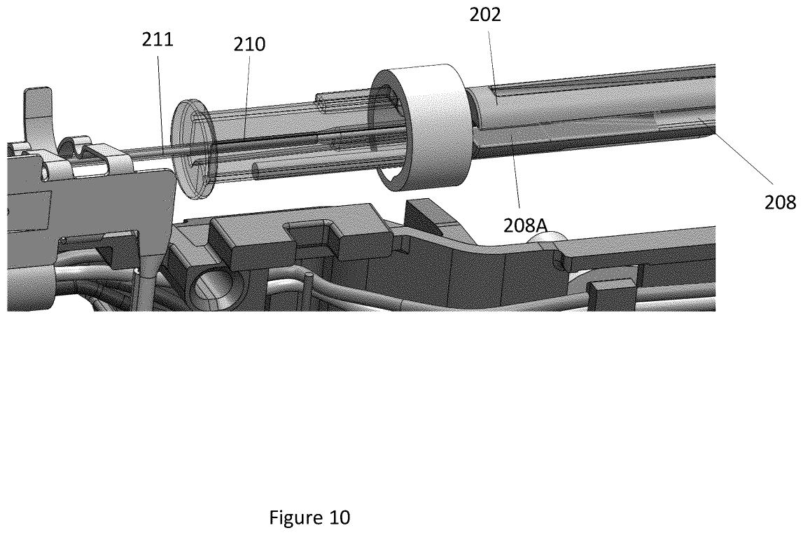

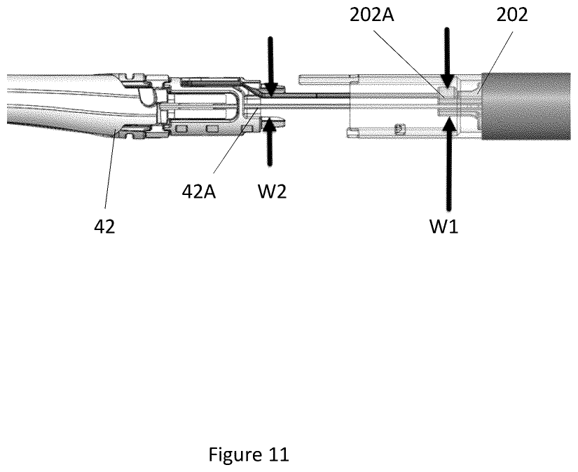

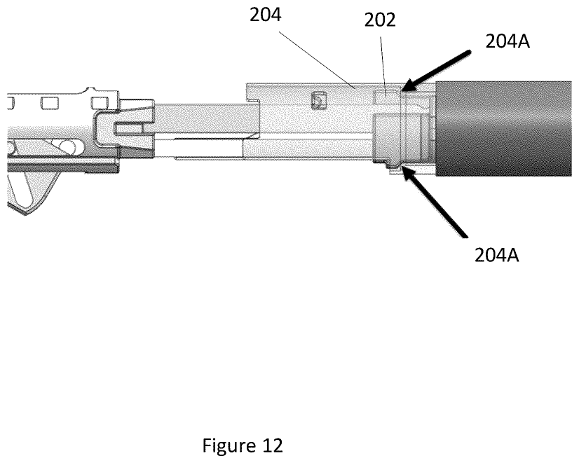

For example, as seen in FIGS. 7-12, the singular inner guide 202 can include an active rod channel 208, which can define space within the shaft 30 through which an active rod 207 can be disposed. The active rod channel 208 is depicted as being located on a lateral portion of the singular inner guide 202. The active rod 207 can be used to transmit energy from the handpiece 20 to the end effector 40, which can be required for electrical operations involving the instrument 10. Generally, the active rod 207 can be a conductor extending between the proximal end (or the handpiece 20) and the end effector, and can be configured to conduct electricity generated by a component in electrical communication with the proximal end (or the handpiece 20 itself) to grasped tissue in order to treat (e.g., cauterize) the tissue. Beyond providing a space in which the active rod 207 is positioned, the active rod channel 208 can assist in isolating and insulating the active rod 207 from other components running through the shaft 30. In some variations, bipolar energy may be desired, and the active rod channel 208 may be comprised of more than one active rod channel to contain components of the circuit responsible for two polarities of energy. FIG. 7 depicts a distal end of the instrument 10, and the active rod 207 can be seen entering into the end effector 40. FIG. 8 depicts a proximal end of the singular inner guide 202, which includes an additional feature of the active rod channel 208. The distal end of the inner guide 202 can guide the active rod 207 during installation so as not to damage the active rod 207 or its insulation. The active rod channel 208 can include a ramp 208A that slopes inwardly toward a center of the singular inner guide 202 so that the active rod 207, when seated within the active rod channel 208, exits the proximal end of the singular inner guide 202 approximately along the longitudinal axis of the single inner guide 202. This ramp 208A can improve the electrical connection between components in the handpiece 20 and the active rod 207. Further, as seen in FIGS. 9 and 10, the shaft 30 can include an adapter portion 210, which can assist in connecting the active rod 207 to a proximal contact 211 located within the handpiece 20 after the active rod 207 has been transitioned to generally align with the central longitudinal axis via the ramp 208A. Also included in FIG. 9 is a tapered alignment hole 212 positioned within each of the singular inner guide 202 and the monolithic outer tube 204. Alignment between the singular inner guide 202 and the monolithic outer tube 204 can be confirmed by inserting an element into the tapered alignment hole 212 to contact each element therethrough. This insertion can confirm rotational and/or longitudinal alignment between the pieces during manufacturing, for example. During assembly, rotational alignment is driven by a width W1 of a distal tab 202A of the inner guide 202 to a width W2 of a proximal gap 42A of the jaw 42, which can be seen in FIG. 11. Axial alignment is driven by a distal end of the inner guide 202 stopping against a counterbore 204A of the outer tube 204, which can be seen in FIG. 12.

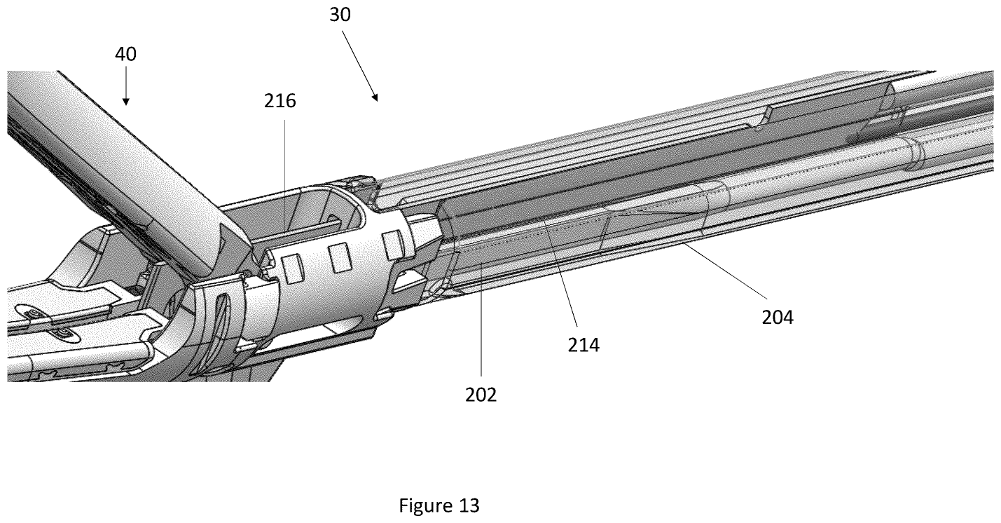

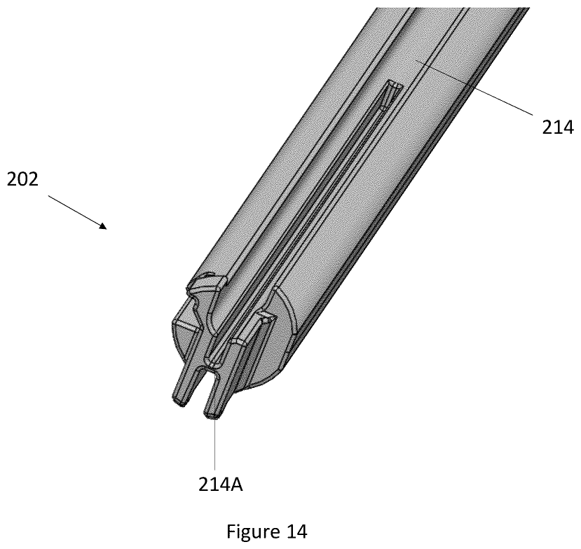

FIGS. 13 and 14 depict a distal knife track 214 defined within the singular inner guide 202, which can be used to support a knife 216 or other blade used for cutting tissue. The knife track 214 can include a distal sub-channel 214A that is deeper and more narrow than the main knife track 214. In operation, actuation of one of the inputs on the handpiece 20, such as the trigger activation 26, can cause the knife 216 to be extended distally through a deliberate gap within the jaws 42 of the end effector 40 in an activation stroke. The distal knife track 214 can provide a pathway through which the knife 216 can be fired, as well as provide support for the knife 216 to prevent buckling in the event of a misfire or an encounter with tough tissue, or the like. The distal sub-channel 214A can provide further support to the knife 216, and the distal knife track, in general, can prevent buckling of the knife 216 when the knife 216 is activated to transect grasped tissue.

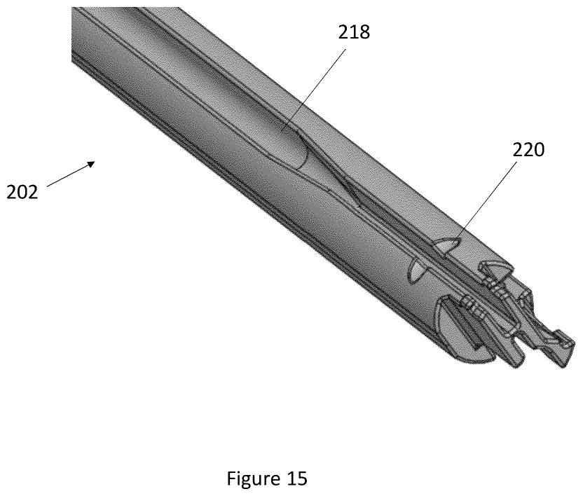

FIG. 15 depicts a similar feature, a closure band channel 218, which can be used to prevent bucking in a closure band (not shown). The closure band can be moved longitudinally along the shaft 30 to effect movement in the jaws 42 of the end effector 40. A dedicated space for the closure band such as the closure band channel 218 can reduce the risk of damage to the closure band, and, in turn, reduce the risk of operative failure in the instrument 10 generally. The closure band channel 218 can be located directly opposite the distal knife track 214, although as mentioned above, the specific location of the various channels, including the distal knife track 214 and the closure band channel 218 can vary. The closure band channel 218 can taper toward a distal end of the single inner tube 202 to accommodate a more narrow component of the closure band. Also depicted in FIG. 15 and running laterally across a portion of the closure band channel 218 is an assembly fixture support groove 220.

The shaft 30 can operate a frame or skeleton through which inputs can travel to effect operations at the end effector. These inputs can be carried by elements such as the active rod 207 and/or the knife 216. However, the shaft 30 itself, as described herein, can consist generally of the single inner tube 202 and the monolithic outer tube 204. As explained, the single inner tube 202 can include a plurality of channels meant to house or guide the various elements, but those elements themselves can be generally described as being separate from the shaft 30, despite being supported or carried by the shaft 30.

As will be appreciated by a person skilled in the art, electronic communication between various components of a robotic surgical system can be wired or wireless. A person skilled in the art will also appreciate that all electronic communication in the system can be wired, all electronic communication in the system can be wireless, or some portions of the system can be in wired communication and other portions of the system can be in wireless communication.

The systems, devices, and methods disclosed herein can be implemented using one or more computer systems, which may also be referred to herein as digital data processing systems and programmable systems.

A computer system can also include any of a variety of other software and/or hardware components, including by way of non-limiting example, operating systems and database management systems. Although an exemplary computer system is depicted and described herein, it will be appreciated that this is for sake of generality and convenience. In other embodiments, the computer system may differ in architecture and operation from that shown and described here.

Preferably, components of the invention described herein will be processed before use. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high energy electrons. The radiation kills bacteria on the instrument and in the container. The sterilized instrument can then be stored in the sterile container. The sealed container keeps the instrument sterile until it is opened in the medical facility.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An electrosurgical device, comprising:
a proximal end;
a shaft extending distally from the proximal end, the shaft consisting of:
a monolithic outer tube, and
a single inner guide disposed within the monolithic outer tube and extending a length thereof;
an outer guide coupled to the shaft and the proximal end, the outer guide being configured to minimize unwanted motion between the proximal end and the shaft; and
an end effector operatively coupled to a distal end of the shaft, the end effector comprising a pair of jaws configured to grasp tissue and to receive a blade from the shaft to transect the grasped tissue.

2. The electrosurgical device of claim 1, wherein the singular inner guide includes a plurality of channels defined in an exterior thereof, each of the channels within the plurality of channels extending an entire length of the singular inner guide and being configured to receive an element in electrical communication with the proximal end.

3. The electrosurgical device of claim 2, wherein the plurality of channels includes an active rod channel configured to receive an active rod therein, the active rod channel including a proximal ramp feature configured to direct the active rod received within the active rod channel toward a center of the singular inner guide.

4. The electrosurgical device of claim 2, wherein the plurality of channels includes a knife track being configured prevent buckling of a knife positioned therein during an activation stroke of the knife.

5. The electrosurgical device of claim 1, wherein the monolithic outer tube has a tapered hole therein and the singular inner guide has a second tapered hole therein, and wherein the first and second tapered holes are configured to align when the singular inner guide is properly positioned within the monolithic outer tube.

6. The electrosurgical device of claim 1, further comprising heat shrink tubing disposed around the shaft.

7. The electrosurgical device of claim 1, wherein the monolithic outer tube includes a central lumen configured to receive the shaft therethrough.

8. The electrosurgical device of claim 1, wherein the outer guide includes an outer guide clip configured to couple to the outer guide and to the shaft, and configured to prevent rotation of the shaft relative to the monolithic outer guide.

9. The electrosurgical device of claim 8, wherein the outer guide clip is substantially Y-shaped.

10. The electrosurgical device of claim 8, wherein the outer guide clip is configured to couple to corresponding grooves in an exterior of the shaft.

11. An electrosurgical device, comprising:
a proximal end;
a shaft extending distally from the proximal end and being secured to the proximal end via an outer guide including an outer guide base, an outer cover, and an outer guide clip configured to couple the outer guide to the shaft, the shaft consisting of:
a monolithic outer tube, and
a single inner guide disposed within the outer tube; and
an end effector operatively coupled to a distal end of the shaft, the end effector comprising a pair of jaws configured to grasp tissue,
wherein the outer guide is configured to minimize user independent motion between the proximal end and the shaft.

12. The electrosurgical device of claim 11, wherein the singular inner guide includes a plurality of channels defined in an exterior thereof, each of the channels within the plurality of channels extending an entire length of the singular inner guide and being configured to receive an element in electrical communication with the proximal end.

13. The electrosurgical device of claim 12, wherein the plurality of channels includes an active rod channel defined in an exterior thereof, the singular inner guide including a proximal ramp feature configured to direct an active rod received within the active rod channel toward a center of the singular inner guide.

14. The electrosurgical device of claim 12, further comprising an active rod extending between the handpiece and the end effector, and being configured to conduct an electrical current generated by the handpiece to tissue grasped in the pair of jaws.

15. The electrosurgical device of claim 12, further comprising a knife disposed in the plurality of channels and being configured to extend into the pair of jaws to transect grasped tissue during an activation stroke triggered by an input at the proximal end.

16. The electrosurgical device of claim 12, wherein the plurality of channels includes a knife track being configured prevent buckling of a knife positioned therein during an activation stroke of the knife.

17. The electrosurgical device of claim 11, wherein the monolithic outer sleeve has a tapered hole therein and the singular inner guide has a second tapered hole therein, and wherein the first and second tapered holes are configured to align when the singular inner guide is properly positioned within the monolithic outer sleeve.

18. The electrosurgical device of claim 11, further comprising heat shrink tubing disposed around the shaft.
